SEQUENCE LISTING (10 sequences)

SEQ ID NO: 1 - protein, 20 aa, Helicobacter pylori
MLVTKLAPDFKAPAVLGNNE

SEQ ID NO: 2 - protein, 20 aa, Helicobacter pylori
MKTFEILKHLQADAIVLFML

SEQ ID NO: 3 - protein, 20 aa, Helicobacter pylori
NVKGTDFFNVHKATEEIYEE

SEQ ID NO: 4 - protein, 4 aa, Helicobacter pylori
KDTP

SEQ ID NO: 5 - protein, 21 aa, Helicobacter pylori
IVLFMKVHDFHWNVKGTDFFN

SEQ ID NO: 6 - protein, 46 aa, Helicobacter pylori
MKTFEILKHLQADAIVLFMKVHNFHWNVKGTDFFNVHKATEEIYEE

SEQ ID NO: 7 - protein, 33 aa, Helicobacter pylori
GAARACGGCCGARATIYTIAARCAYYTICARGC

SEQ ID NO: 8 - protein, 24 aa, Helicobacter pylori
TCYTCGGCCGTYTCYTCIGTNGCY

SEQ ID NO: 9 - protein, 26 aa, Helicobacter pylori
RATIYTCGGCCGYYICARGCIGAYGC

SEQ ID NO: 10 - protein, 23 aa, Helicobacter pylori
ATYTCGGCCGTIGCYTTRTGNAC

(19) United States
(12) Patent Application Publication
(10) Pub. No.: US 2001/0010821 A1
(43) Pub. Date: Aug. 2, 2001

(54) HELICOBACTER PROTEINS AND VACCINES

(76) Inventors: DERMOT KELLEHER, DUBLIN (IE); HENRY WINDLE, DUBLIN (IE); WILLIAM BYRNE, DUBLIN (IE); ROSS MCMANUS, DUBLIN (IE)

Correspondence Address: JACOBSON PRICE HOLMAN & STERN, THE JENIFER BUILDING, 400 SEVENTH STREET NW, WASHINGTON, DC, 20004

(21) Appl. No.: 08/775,765

(22) Filed: Dec. 31, 1996

Related U.S. Application Data
(63) Continuation of application No. PCT/IE95/00036, filed on Jul. 3, 1995.

(30) Foreign Application Priority Data
Jul. 1, 1994 (IE) 94 0538
Apr. 6, 1995 (IE) 95 0249

(51) Int. Cl. A61K 39/02

(52) U.S. Cl. 424/234.1; 424/236.1

(57) ABSTRACT

A vaccine includes at least one Helicobacter, especially Helicobacter pylori protein to which immunoreactivity is detected in H. pylori negative individuals. The Helicobacter proteins are preferably less than 30 kDa and the vaccine especially includes 24 to 25 kDa and/or 18 to 19 kDa proteins. The vaccine may include interleukin 12 as an adjuvant.

### FIELD OF THE INVENTION

The invention relates to a vaccine or therapeutic composition for the treatment or prophylaxis of Helicobacter pylori associated disease and protein used in the vaccine.

### BACKGROUND

Helicobacter pylori is a widely prevalent organism found on gastric biopsy in approximately 30% of the population less than 40 years old with increasing incidence thereafter. The organism is a causative agent of chronic gastritis in humans (e.g. Marshall & Warren 1984¹; Blaser, 1990²). Epidemiological studies have shown that H. pylori is most commonly found in association with gastritis. Serological investigations have demonstrated that evidence of a current or prior infection can be found in 30-50% of a randomly chosen population of blood donors. No direct causal relationship has been conclusively proven for duodenal ulcer disease. However, the organism is found in 95% of patients with duodenal ulcer. Furthermore, eradication of the organism results in rapid ulcer healing (e.g. Rauws & Tytgat, 1990³). These data provide strong evidence that H. pylori is a dominant factor in the development of duodenal ulcer. Additional evidence for the pathogenic involvement of H. pylori in these conditions has been provided by studies with gnotobiotic piglets (Lambert et al., 1987⁴) and the fulfilment of Koch's postulates with human volunteers (Marshall et al., 1985⁵; Morris & Nicholson, 1987⁶).

In addition, there is now strong circumstantial evidence implicating H. pylori in the pathogenesis of gastric carcinoma (e.g. Jiang et al., 1987⁷; Lambert et al., 1986⁸; Crabtree et al., 1992⁹; 1993¹⁰; Forman et al., 1990¹¹, 1991¹²; Nomura et al., 1991¹³; Parsonnet et al., 1991¹⁴). Most recently, the Eurogast Study Group, led by Forman (1993¹⁵), demonstrated a significant relationship between H. pylori seropositivity and gastric cancer mortality and incidence. Indeed, there is now a convincing body of literature implying infection with H. pylori in a considerable proportion of upper gastrointestinal morbidity. A number of hypotheses have been suggested for the pathogenic mechanisms of H. pylori induced gastroduodenal disease, including the production of cytotoxins and mechanical disruption of the epithelium (e.g. Blaser, 1992¹⁶). Interestingly, however, many infected persons remain asymptomatic despite the persistent presence of the pathogen (Taylor & Blaser, 1991¹⁷).

### STATEMENTS OF INVENTION

According to the invention, there is provided a vaccine including at least one Helicobacter protein or derivative or fragment or precursor or mutant thereof to which immunoreactivity is detected in H. pylori negative individuals. Preferably the immunoreactivity is antibody based.

In a preferred embodiment of the invention, the protein is a Helicobacter pylori protein.

In a preferred embodiment of the invention the protein has a molecular weight of less than 30 kDa, especially less than 29 kDa, particularly less than 28 kDa and ideally less than 27 kDa.

In a particularly preferred embodiment of the invention, the vaccine -includes a 24 to 25 kDa protein or a derivative or fragment or precursor or mutant thereof.

The 24 to 25 kDa protein is further characterised in that it has a N-terminal amino acid sequence listed in Sequence Id. No. 2, or a portion thereof.

The 24 to 25 kDa protein is further characterised in that it has an internal amino acid sequence listed in Sequence Id. No. 4, or a portion thereof.

In a particularly preferred embodiment of the invention, the vaccine includes an 18 to 19 kDa protein, or a derivative, fragment or precursor or mutant thereof.

Most preferably the 18 to 19 kDa protein has a N-terminal amino acid sequence listed in Sequence Id. No. 1, or a portion thereof.

The 18 to 19 kDa protein also includes an internal amino acid sequence listed in Sequence Id. No. 3, or a portion thereof.

In a particularly preferred embodiment of the invention, the 18 to 19 kDa protein has an N-terminal Sequence listed in Sequence Id. No. 6, or a portion thereof.

The vaccine may include a pharmaceutically acceptable carrier.

The vaccine may be combined with a suitable adjuvant such as interleukin 12 or a heat shock protein or both.

The vaccine may include at least one other pharmaceutical product such as an antibiotic and/or anti-bacterial agent such as bismuth salts. Typically the antibiotic is selected from one or more of metronidazole, amoxycillin, tetracycline, erythromycin, clarithromycin or tinidazole.

The vaccine may be in a form for oral, intranasal, intravenous or intramuscular administration.

The vaccine may include a peptide delivery system.

The vaccine is ideally for the treatment or prophylaxis of Helicobacter pylori infection or Helicobacter pylori associated disease(s).

According to another aspect of the invention there is provided a Helicobacter protein or derivative or fragment or precursor or mutant thereof to which immunoreactivity is detected in H. pylori negative individuals. Preferably, the immunoreactivity is antibody based.

Preferably the Helicobacter pylori is a Helicobacter pylori protein.

In a preferred embodiment of the invention, the protein has a weight of less than 30, especially less than 29, particularly less than 28 and ideally less than 27 kDa.

In a particularly preferred embodiment of the invention, the Helicobacter pylori protein is a 24 to 25 kDa protein or derivative or fragment or precursor or mutant thereof.

The 24 to 25 kDa Helicobacter pylori protein is characterised in that it includes the N-terminal amino acid sequence listed in Sequence Id. No. 2, or a portion thereof.

The 24 to 25 kDa Helicobacter pylori protein is further characterised in that it includes an internal amino acid sequence listed in Sequence Id. No. 4, or a portion thereof.

In another preferred embodiment of the invention, the Helicobacter pylori is an 18 to 19 kDa protein or derivative or fragment or precursor or mutant thereof.

The 18 to 19 kDa Helicobacter pylori is characterised in that it includes the N-terminal amino acid sequence listed in Sequence Id. No. 1, or a portion thereof.

The 18 to 19 kDa Helicobacter pylori is further characterised in that it includes the internal amino acid sequence listed in Sequence Id. No. 3, or a portion thereof.

The 18 to 19 kDa Helicobacter pylori is further characterised in that it includes the N-terminal amino acid sequence listed in Sequence No. 6.

The invention also provides a method for the treatment or prophylaxis of Helicobacter pylori associated disease in a host, comprising administering to the host an immunologically effective amount of one or more of the Helicobacter proteins of the invention.

Preferably, the Helicobacter pylori protein is administered in combination with at least one other pharmaceutical agent.

In a preferred embodiment, the pharmaceutical agent is an antibiotic.

Ideally, the antibiotic is selected from one or more of metronidazole, amoxycillin, tetracycline or erythromycin, clarithromycin, tinidazole.

Typically the pharmaceutical agent includes an antibacterial agent such as bismuth salts.

In a preferred embodiment of the invention an adjuvant is administered in combination with the Helicobacter protein. Preferably the adjuvant is interleukin 12 or a heat shock protein or both.

The invention also provides the use of one or more Helicobacter proteins of the invention for the preparation of a medicament for the treatment or prophylaxis of Helicobacter pylori associated disease(s).

The invention further provides monoclonal or polyclonal antibodies or fragments thereof, to the proteinaceous material of the invention and purified antibodies or serum obtained by immunisation of an animal with the vaccine according to the invention.

The invention also provides the use of such serum and antibodies in the treatment or prophylaxis of Helicobacter associated disease(s) and in particular Helicobacter pylori associated disease(s).

The invention also provides a vaccine for the treatment or prophylaxis of Helicobacter pylori associated disease comprising an immunogenically effective amount of the 24 to 25 kDa Helicobacter pylori protein and/or the 18 to 19 kDa Helicobacter pylori protein of the invention, an adjuvant such as Interleukin 12, and an antibiotic.

The vaccine may include an antibacterial agent such as bismuth salts.

The invention also includes the use of interleukin 12 in combination with the 18 to 19 kDa protein, the 24 to 25 kDa or any other H. pylori subunit as an adjuvant therapy.

Therefore, in another aspect, the invention provides a vaccine against H. pylori comprising an immunogenically effective amount of a Helicobacter or a subunit, fragment, derivative, precursor or mutant thereof in combination with interleukin 12 as an adjuvant. Preferably the Helicobacter is Helicobacter pylori.

In one embodiment of the invention the vaccine includes an antibiotic and may alternatively or additionally include an antibacterial agent.

### DESCRIPTION OF DRAWINGS

FIG. 1: Adult sera (CLO negative) screened for the presence of anti-H. pylori IgG antibodies. The figure shows a Western blot of H. pylori probed with serum obtained from CLO negative individuals. All sera were diluted 1:100 in PBS containing fat-free dried skimmed milk (5%, w/v). Proteins were transferred from SDS-PAGE gels to PVDF membrane. The antigen-antibody complexes were detected on washed membranes using an enhanced chemiluminescent detection system. Each track represents a different serum sample.

FIG. 2: Absorbed sera : Sera from two individuals negative for H. pylori were absorbed with either whole C. jejuni (track A), H. pylori (track B), or E. coli (track C).

FIG. 3: Partial purification of 18 and 25 kDa proteins: Both proteins were purified from whole Helicobacter pylori on the basis of molecular weight using preparative continuous-elution SDS-PAGE on a Model 491 Prep-Cell (Bio-Rad).

FIG. 4: Sera obtained from CLO negative children screened for the presence of anti-H. pylori IgG antibodies. The figure shows a Western blot of H. pylori probed with serum obtained from CLO negative children. All sera were diluted 1:50 in PBS containing fat-free dried skimmed milk (5%, w/v). Each track represents a different serum sample.

FIG. 5: Antigens recognised on C. jejuni and E. coli by anti-H. pylori antiserum. The figure shows a Western blot of H. pylori (track A), C. jejuni (track B) and E. coli (track C) probed with rabbit anti-H. pylori antiserum. Each bacterium (5 gg) was subjected to SDS-PAGE followed by immunoblotting.

FIG. 6: Western blot of purified 25 kDa protein developed with serum from an individual negative for H. pylori. Purified 25 kDa protein was subjected to SDS-PAGE and Western blotting. The blot was probed with serum obtained from a subject uninfected with H. pylori.

FIG. 7: Biotinylation of proteins located on the surface of Helicobacter pylori. Agar-grown H. pylori were harvested in phosphate buffered saline (pH 7.3) and washed twice in this buffer prior to biotinylation of surface exposed proteins. Bacteria (^{˜}2 mg ml⁻¹) were resuspended in PBS (1 ml) and prewarmed to 37° C. Thereafter, biotin-X-NHS (Sulfosuccinimidyl-6(biotinamido)-hexanoate; Calbiochem) was added to a final concentration of 1 IM and was prepared immediately before use. After mixing to 10 min at 37° C., the labelling reaction was terminated by the addition of 1.5 M Tris-Cl (pH 8) to a final concentration of 10 mM. The suspension was washed three times by centrifugation (10,000 g, 1 min) in ice-cold PBS. Examination of the bacteria by light microscopy after the labelling and washing procedures demonstrated that the cells were still intact and motile. Biotinylated H. pylori was subjected to analytical SDS-PAGE, followed by Western blotting, to identify the biotinylated proteins. The Western blots were developed with Extravidin-peroxidase (Sigma).

FIG. 8 Illustrates thymidine incorporation of lymphocytes in response to H. pylori in the presence and absence of interleukin 12.

FIG. 9: Illustrates thymidine incorporation of peripheral blood mononuclear cells in the presence or absence of H. pylori with or without anti-interleukin 10 or recombinant interleukin 12.

### DETAILED DESCRIPTION OF THE INVENTION

We have studied the prevalence of immuno-reactivity to H. pylori in both infected and un-infected individuals and found that un-infected individuals have a high response to H. pylori both in their B-cell and T-cell systems. Specifically, the T-cell immune response to H. pylori seems to be stronger in individuals who are negative for the organism. In this regard we have examined the secretion of the cytokine-interferon which is extremely important for the killing of microorganism by macrophages. Secretion of -interferon by T-cells of patients infected with H. pylori was considerably less than secretion by un-infected individuals when their T-cells were exposed to the organism (Fan et al., 1993¹⁸). Hence, these data suggest that individuals who are H. pylori negative have been exposed to the organism and may potentially have cleared the organism. Furthermore, the response to the organism is considerably more potent in this group of individuals than it is in the H. pylori positive patients.

The term “H. pylori negative individuals” means individuals with immunoreactivity to H. pylori who do not have evidence of H. pylori gastric colonisation as determined by techniques such as one or more of rapid urease testing, histological examination or culture of gastric biopsies.

A second component relates to the antibody response to H. pylori in H. pylori negative individuals. Briefly, we have demonstrated using a sensitive detection system that the majority of H. pylori negative individuals have detectable antibodies to two H. pylori proteins. Specifically, these proteins are of MW 18-19 and 24-25 kDa. It is thus proposed that a potent immune response to these antigens results in protective immunity to the organism. Furthermore, we have partially sequenced these proteins.

In many cases antibodies to H. pylori are detected by ELISA.

An inherent constraint in the design of ELISA based detection systems is that of establishing a cut off point such that all samples below this threshold are considered negative. Clearly, many seropositive cases will remain undetected in this situation and a true estimate of the incident of prior contact with the organism will thereby be underestimated. In this approach we use Western blotting to investigate antigen specificity of systemic responses to H. pylori in both healthy and H. pylori-infected individuals and shown that the incidence of seropositivity in H. pylori negative individuals is much greater than has previously been demonstrated. Furthermore, we have demonstrated that antibodies to a 24 to 25 kDa protein are detectable in the majority of H. pylori negative individuals. These were detected using a technique which we have modified called Enhanced Chemiluminescence. Enhanced Chemiluminescence on Western blot analysis reveals that the majority of uninfected individuals have antibodies which are specific for H. pylori and recognise antigens which are not present on other micro organisms. Of these antigens the most common one recognised is a 24 to 25 kDa protein which appears to be specific to H. pylori. Hence, these data suggest that immunisation with the 24 to 25 kDa protein or sub-unit thereof could have the potential to confer protective immunity on individuals who are either un-infected with the organism or individuals in whom the organism has been cleared by anti-bacterial treatment. A second protein was also identified at 18 to 19 kDa in a large subgroup of H. pylori negative individuals. Similarly, immunization with this protein or subunit thereof could also confer protective immunity.

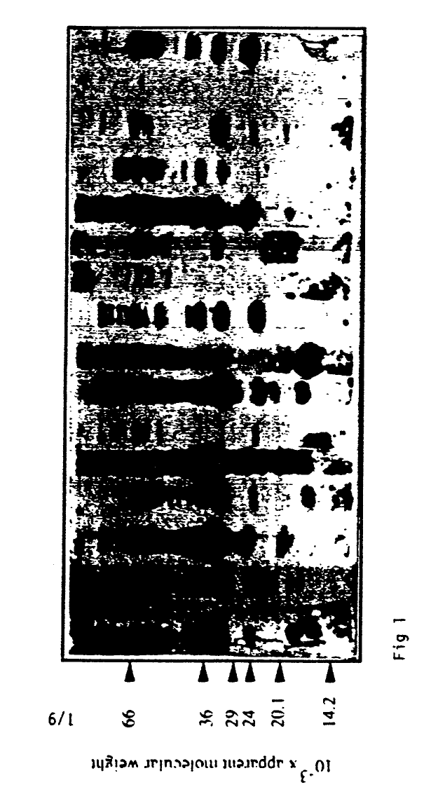

We have developed a novel assay for detection of antibodies to H. pylori. This assay uses Western blotting and Enhanced Chemiluminescence (ECL). Using this assay we have demonstrated that approximately 75% of individuals who are negative for H. pylori by routine testing such as the rapid urease test have in fact got detectable antibodies to H. pylori (FIG. 1).

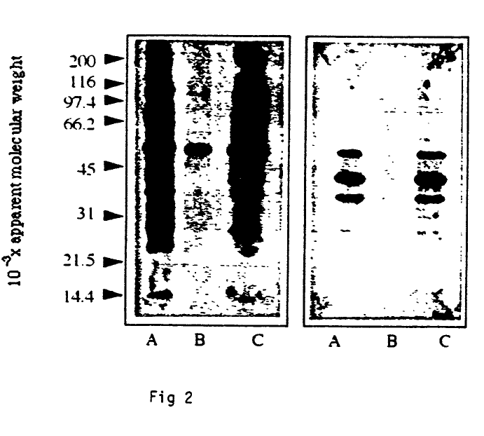

Furthermore, these antibodies are not absorbed by C. jejuni or by E. coli suggesting that this is a specific antibody response (FIG. 2). Of particular note we have performed characterisation of the antigens recognised by these antibodies by molecular weight, using ECL Western blotting. Sera from un-infected individuals recognize a range of antigens on H. pylori. The most common antigen recognised is a 24 to 25 kDa protein which is recognised in over 70% of individuals who are negative for the organism on Rapid urease testing. Hence this suggests that the 24 to 25 kDa protein may be an immunodominant antigen which evokes a powerful immune response in individuals who are negative for the organism. A second protein was identified at 18 to 19 kDa which elicited significant antibody responses in H. pylori-negative children. These proteins have been further characterised by N-terminal and internal sequencing as outlined in the Appendix.

Finally a cytokine produced by macrophages called interleukin 12 may significantly enhance γ-interferon production in response to antigen. As stated previously, antigen-specific interferon production is reduced with H. pylori positive individuals. The addition of IL-12 to immunisation schedules with a 25 CkDa protein would be expected to boost host immunity to H. pylori by augmenting the γ-interferon response.

Materials.

All antibodies were obtained from Dako Ltd., High Wycombe, Bucks., U.K. All other chemicals and solvents were obtained from either the Sigma Chemical Company Ltd., Poole, Dorset, United Kingdom or BDH Chemicals Ltd., Poole, Dorset, United Kingdom.

SDS-PAGE.

Discontinuous SDS-PAGE was performed essentially as described by Laemmli (1970)19. A total of 5 mg of acetone-precipitated H. pylori protein were located into each well. Gels were either stained with Coomassie Blue R-250 or processed for immunoblotting. Broad range molecular weight markers were purchased from Bio-Rad Laboratories, 3300 Regatta Blvd., Richmond, Calif. 94804. The molecular masses are expressed as kDa.

Western Blotting.

Proteins from SDS-PAGE gels (30% T/2.67% C) were electroblotted (0.8 mA/cm² for 1 h) to PVDF membrane using a semi-dry blotting apparatus (LKB/Pharmacia), essentially as described by Towbin et al, (1979). Primary antibodies (human serum; 1/50-1/100 dilution) were detected using a 1/5,000 dilution of anti-human IgG (horseradish peroxidase-conjugated) in combination with enhanced chemiluminescence. Blots were washed in PBS containing fat-free dried skimmed milk (5%, w/v) and Tween-20 (0.05%, v/v). Blots were exposed to Kodak X-OMAT S film for 1-10 s. Exposed films were developed in Kodak LX-24 developer and fixed in Kodak dental X-ray fixer.

Sera.

Serum samples were obtained from the Research Centre, Our Ladies Hospital for Sick Children, Crumlin, Dublin. All subjects were attended for medical conditions other than gastroenterological disorders. In addition, blood samples were obtained from a randomly selected cohort of children (Harcourt Street Childrens Hospital, Dublin) or from adults attending the gastenterology unit at St. James's Hospital, Dublin. All patients had a rapid urease (CLOtest) performed. Patients were defined as H. pylori positive or negative on the basis of positive or negative responses on rapid urease test.

Anti-H. pylori antiserum. Anti-H. pylori antiserum was a kind gift from Prof. B. Drumm and Dr. M. Clyne. The antiserum was raised in New Zealand white rabbits against whole H. pylori using conventional immunizing and boosting procedures.

Protein Measurements.

Protein was measured by the method of Markwell et al. (1978)²⁰ with bovine serum albumin as the protein standard.

Absorption of Sera.

Antisera were absorbed with either E. coli or C. jejuni by incubating a suspension of the bacteria with patient sera for 2 h at room temperature with gentle mixing. The bacteria were removed from suspension by centrifugation (12,000×g, 3 min).

Bacterial Strains and Growth Conditions.

The clinical isolates H. pylori used in this study were isolated from antral biopsies obtained from patients attending the gastroenterology clinic at St. James's Hospital, Dublin. H. pylori was grown under microaerophilic conditions for 4 days on 7% lysed horse blood agar at 37° C. Cells were harvested into ice-cold phosphate buffered saline (pH 7.5) containing PMSF (1 mM), EDTA (1 mM), and leupeptin (50 μg/ml). The cells were washed twice by centrifugation (10,000×g, 5 min, 4° C.) in this buffer before use. C. jejuni was a clinical isolate from stool in a patient with C. jejuni enteritis and was grown for two days exactly as described above with the exception that the incubation temperature was 42° C. The strain of E. coli used in this study is commercially available (Gibco) NTCC 11637 and was kindly provided by Dr. Ciaran Cronin, Dpt. Pharmacology, University College Dublin.

Methods Used in the Identification and Partial Purification of Two Novel Antigens from Helicobacter pylori

Methods

Western Blotting.

Proteins from SDs-PAGE gels (30% T/2.67% C) were electroblotted (0.8 mA/cm² for 1 h) to PVDF membrane using a semi-dry blotting apparatus (LKB/Pharmacia). Primary antibodies (human serum; 1/50-1/100 dilution) were detected using a 1/5,000 dilution of anti-human IgG (horseradish peroxidase-conjugated) in combination with enhanced chemiluminescence (see below). Blots were washed in phosphate buffered saline (pH 7.5) containing fat-free dried skimmed milk (5%, w/v) and Tween-20 (0.05%, v/v). Blots were exposed to Kodak X-OMAT S film for 1-10 s. Exposed films were developed in Kodak LX-24 developer and fixed in Kodak dental X-ray fixer.

Enhanced Chemiluminescence (ECL)

The use of chemiluminescence to detect antibodies in Western blotting in preference to the conventional procedures of employing chromogenic substrates as detection reagents was adopted primarily because of the reporting gain in the sensitivity of detection (approximately 10-fold) over that found when chromogens are used. Oxidized luminol emits visible light and the intensity of this light emission is increased 1000-fold in the presence of chemical enhancers (e.g. iodophenol). The method is described blow:

Substrate Concentration/Amount

Luminol 1.2 mM (in 0.1 M-Tris (50 ml), pH 8.8)

4-Iodophenol 0.4 mM (dissolved in DMSO before use)

Hydrogen Peroxide 17 p1 of a 30% (v/v) solution

Blots were incubated in the above mixture for one minute and then exposed to X-ray film as described above.

Partial Purification of 18 and 25 kDa Proteins

Both proteins were partially purified from whole Helicobacter pylori on the basis of molecular weight (FIG. 2) using preparative continuous-elution sodium dodecyl sulphate polyacrylamide gel electrophoresis (SDS-PAGE) on a Model 491 Prep-Cell (Bio-Rad). This method enables us to quantitatively purify preparative amounts of proteins in a soluble form.

Purification Method

25 mg H. pylori were precipitated with ice-cold acetone, washed once in acetone and the precipitate then solubilised in 3.8 ml SDS-PAGE sample buffer (62 mM Tris, pH 6.8; glycerol (10%, v/v); SDS (2%, v/v); 2-mercaptoethanol (5%, v/v); bromophenol blue (0.002%, v/v). Published electrophoretic procedures, with very minor modifications, were followed throughout sample preparation.

Loading:

The protein mixture, in sample buffer, was loaded onto a 12.5% polyacrylamide tube gel (30% T/2.67% C). The dimensions of the tube gel were: 28 mm internal diameter; upper surface 3.6 cm²; stacking gel 2 cm; resolving gel 10 cm.

Running Conditions:

Electrophoresis was performed at 40 mA (constant current) overnight at room temperature. Fractions (1 ml) were collected at 0.1 ml/min. Samples of each fraction (5 μl) were subjected to analytical SDS-PAGE to assess the purity and antigenicity of each protein. Every fraction within the molecular mass region of interest was screened by both SDS-PAGE (to assess purity) and Western blotting (to assess antigenicity) in an attempt to isolate and characterise the individual immunogenic proteins. The resolution of this technique is such that pure preparations of single proteins may be achieved once optimal electrophoretic conditions have been established. Preliminary optimization protocols entailed electrophoresing mixtures of H. pylori proteins under conditions designed to favour high resolution of low molecular weight proteins. The final electrophoretic conditions used to achieve partial purification of the selected proteins are detailed in the Methods section. Using these exact conditions the 18 kDa proteins eluted between 11-14 ml and the 25 kDa protein eluted within 16-20 ml. The molecular weights of the proteins were determined by analytical SDS-PAGE using a range of low molecular weight marker proteins (range: 14.5 kDa -66 kDa; code: Sigma SDS-7) and Western blotting confirmed that these proteins were the immunogens of interest.

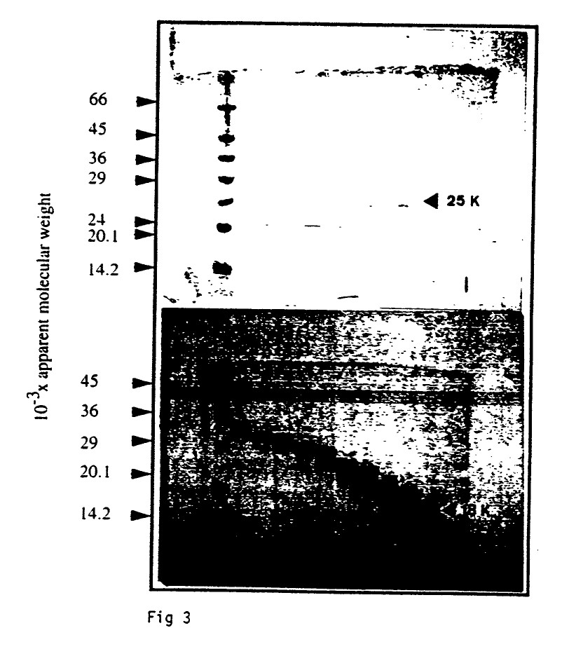

FIG. 1 shows Western blot analysis of antibody responses to H. pylori in individuals negative for H. pylori on Rapid urease testing. Western blotting was performed as previously described using an enhanced chemiluminescence detection system. Antibodies to a large range of H. pylori proteins were seen in individuals who are H. pylori negative on Rapid urease testing. The most common antigen to which an antibody was detected with the 25 kDa protein. FIG. 3 shows a preparative SDS gel elution profile of the 25 kDa and 18 kDa proteins. These proteins have been further characterised by N-terminal and internal sequencing as outlined in the Appendix.

### EXAMPLE 1

CLO Negative Adults

Similarly, a cohort of 19 adult sera was screened for anti-H. pylori IgG antibodies. Each of these subjects was CLO negative, yet 83% had detectable antibodies (IgG) to H. pylori (FIG. 1). Taken together, these data suggest extensive prior contact with H. pylori. The most common antigen to which an antibody was detected was a 25 kDa species.

CLO Negative Children

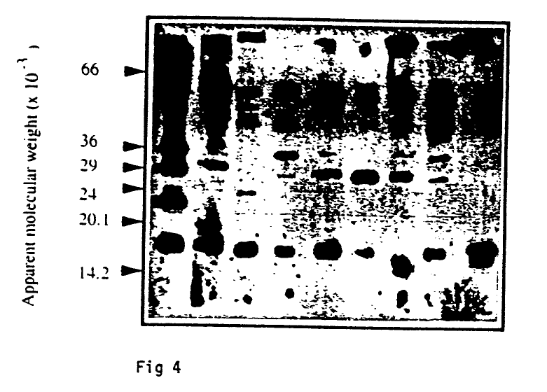

The systemic humoral immune response (IgG) to H. pylori was studied in two groups of children also. None of these subjects had received any form of anti-H. pylori therapy. However, in almost all cases the children had a specific antibody response to H. pylori. The first cohort studies consisted of twenty children (age range: 4-15 years), negative for H. pylori on CLO test. Of these, 75% had detectable IgG antibodies to H. pylori (FIG. 4).

The second cohort of children (n=20) were asymptomatic and presented in hospital with conditions other than gastrointestinal disorders. Yet 13/18 (72%) had detectable IgG antibodies to several H. pylori specific antigens. However, from the intensity of the response the data suggest that the antibody response is most likely due to prior contact with the bacterium, when compared to the considerably stronger response observed with H. pylori positive individuals.

### EXAMPLE 2

Cross Reactivity with other Bacteria

As many bacteria share common antigenic determinants, we examined the extent of cross-reactivity between H. pylori and the closely related C. jejuni, in addition to E. coli, using two complimentary approaches. Firstly, the ability of the anti-H. pylori polyclonal antiserum to recognise antigens on both C. jejuni and E. coli was examined by Western blotting (FIG. 2).

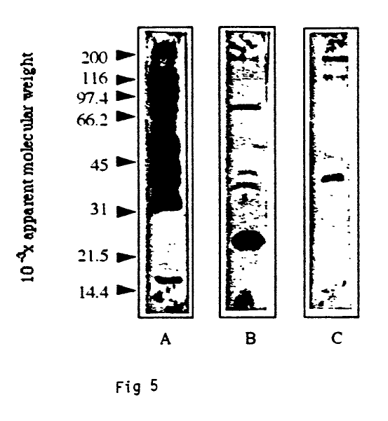

Anti-H. pylori antiserum recognized a number of antigenic determinants on both E. coli and C. jejuni. Specifically, the antiserum recognises proteins of molecular mass 72, 50, 40, 36, and 25 kDa on C. jejuni and proteins of molecular mass 200, 116, 45, and 38 kDa on E. coli (FIG. 5). Of these, only 3 proteins (70, 25 kDa from C. jejuni and 200 kDa from E. coli) show pronounced cross-reactivity with anti-H. pylori antiserum. Therefore, the observed cross reactivity is clearly not extensive. Secondly, absorption experiments demonstrated that this cross reactive antigen recognition was of minor significance. Serum samples absorbed with clinical isolates of H. pylori and C. jejuni in addition to a commercially available strain of E. coli demonstrated that seroreactivity could be eliminated by absorbing with H. pylori but not with C. jejuni or E. coli (FIG. 2). FIG. 2 is a representative experiment. Absorption studies were vperformed on approximately half of the serum samples screened in this study with similar results to those shown. The 18 and 25 kDa proteins were also detected in H. pylori Reference Strains NTCC 11637 and 11638 in addition to all clinical strains tested.

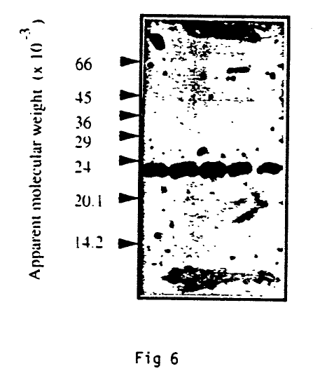

Having partially purified the 26-26 kDa protein by preparation continuous-elution electrophoresis as shown in FIG. 3, we confirmed the antigenicity of the 24-26 kDa protein by probing a Western blot of purified 24-26 kDa protein with serum from an uninfected individual (FIG. 6). The example shown in FIG. 6 is a representative experiment where the blot was incubated with the serum from an H. pylori un-infected individual. Clearly, this serum sample contains antibodies that specifically recognise the 24-26 kDa protein and furthermore, the results of this experiment demonstrate that the antigen preparation is highly enriched for this protein and that no other immunogenic proteins are present in this preparation. We have obtained similar results with the 18-20 kDa protein.

### EXAMPLE 3

Biotinylation of Whole Intact Helicobacter pylori

Agar-grown H. pylori were harvested in phosphate buffered saline (pH 7.3) and washed twice in this buffer prior to biotinylation of surface exposed proteins. Bacteria (^{˜}2 mg ml⁻¹) were resuspended in PBS (1 ml) and prewarmed to 37° C. Thereafter, biotin-X-NHS (Sulfosuccinimidyl-6(biotinamido)-hexanoate; Calbiochem) was added to a final concentration of lmM and was prepared immediately before use. After mixing for 10 minutes at 37° C., the labelling reaction was terminated by the addition of 1.5 M Tris-Cl (pH 8) to a final concentration of 10 mM. The suspension was washed three times by centrifugation (10,000 g, I min) in ice-cold PBS. Examination of the bacteria by light microscopy after the labelling and washing procedures demonstrated that the cells were still intact and motile.

Analysis of Biotinylated Proteins

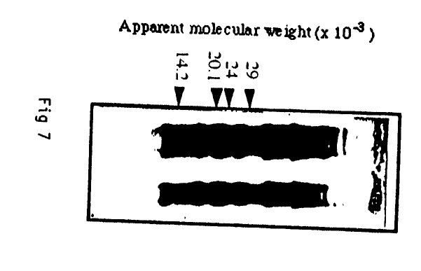

Biotinylated H. pylori was subjected to both analytical and preparative SDS-PAGE, followed by Western blotting, to identify the biotinylated proteins. The Western blots were developed with Extravidin-peroxidase (Sigma). Extensive incorporation of the biotin ester into H. pylori proteins was observed (FIG. 7). Furthermore, it is clear from this figure that proteins in the 18-24 kDa region are biotinylated as are a number of other proteins (Table 1), indicating that these proteins are present on the surface of the bacterium.

TABLE 1

| Biotinylated Protein | Apparent molecular weight |
|---|---|
| 1 | 13,800 |
| 2 | 15,600 |
| 3 | 16,600 |
| 4* | 17,700 |
| 5 | 20,500 |
| 6* | 23,500 |
| 7 | 26,400 |

Method Description

T-cell Immune Response to Helicobacter pylori

We examined the T-lymphocyte proliferative responses to H. pylori using a thymidine incorporation assay. Briefly, lymphocytes were isolated by density gradient centrifugation on a Ficoll-Hypaque gradient. Lymphocytes were seeded into 96-well microtitre plates at a density of 10⁵ cells/well in RPMI 1640 medium containing 10% foetal calf serum. A sonicated irradiated preparation of H. pylori was added at a concentration of 3 μg/ml. Medium alone was added to control wells. In addition interleukin 12 (R&D suppliers) was added at a concentration of 500 pg/ml. Cells were then cultured in a 5% CO₂ incubator for 4 days at 37° C. At 4 days tritiated thymidine 1 μCi/ml was added and cultures continued for a further 24 hours before harvesting using a multiple automated sample harvester. In additional experiments, cells were stimulated using OKT3 antibody to the CD3 T-cell receptor associated complex in the presence and absence of the H. pylori preparation as above. In these studies, interleukin 12 was similarly added. Antibody to interleukin 10 was added in some experiments.

### EXAMPLE 4

T-cell Response to H. pylori is Significantly Augmented by Interleukin 12

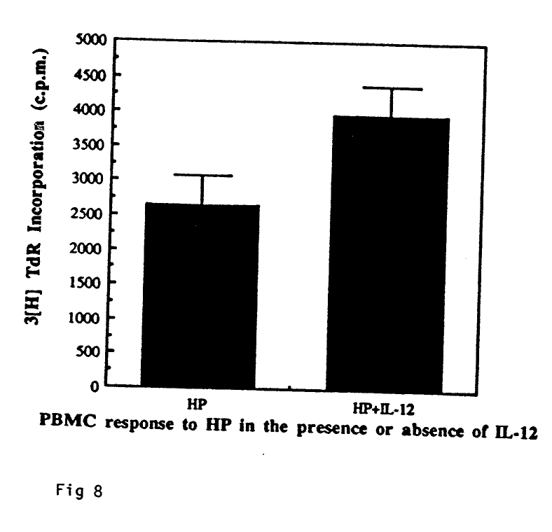

In a cohort of patients in whom lymphocyte proliferative responses to H. pylori were examined as described in the methodology, interleukin 12 significantly increased the proliferation of the peripheral blood mononuclear cell population to H. pylori (n=12, p<0.05) (FIG. 8). These data demonstrate clearly that interleukin 12 has adjuvant properties in respect of H. pylori immunogenicity.

Interleukin 12 Overcomes the Suppression of T-cell Responses induced by H pylori

The H pylori antigen preparation significantly inhibited the proliferation induced by the T-cell mitogen OKT3. This inhibition could be abolished using antibody to interleukin 10, a cytokine produced by T-helper 2 cells known to suppress the T-helper 1 cell pathways involved in cell proliferation. These data therefore suggest that the suppression of T-cell proliferation induced by H pylori is mediated by interleukin 10 through a T-helper 2 pathway. Interleukin 12 also abolished the suppression of T-cell responses induced by H pylori and significantly increased proliferative responses over the baseline OKT3-induced response suggesting that this cytokine is capable of overcoming the effects of the H. pylori T-helper 2 pathway.

FIG. 8 illustrates thymidine incorporation of lymphocytes in response to H. pylori in the presence and absence of interleukin 12. Interleukin 12 significantly augmented proliferation of peripheral blood mononuclear cells in response to H. pylori.

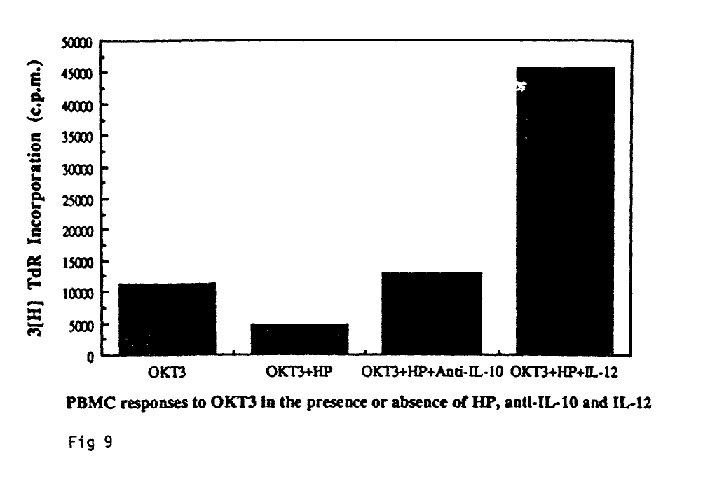

FIG. 9 illustrates thymidine incorporation of peripheral blood mononuclear cells in the presence or absence of H pylori with or without anti-interleukin 10 or recombinant interleukin 12. Both interleukin 12 and anti-interleukin 10 significantly abolished H pylori-induced inhibition of lymphocyte proliferation.

It will be appreciated that interleukin 12 may also be used as an adjuvant with any H. pylori protein or derivative or fragment thereof. Its application is not limited to the specific 25 kDa or 18 kDa proteins referred to above. The interleukin 12 may be conjugated with the H. pylori unit in such a way as to allow the interleukin to be released in vivo, for example by peptic acid and gastric enzymes/or urease.

It will be appreciated by those skilled in the art that while we have referred to a molecular mass of 24 to 25 kDa and 18 to 19 kDa the molecular mass may lie in the 24-26 kDa and 17-19 kDa range. Other related organisms such as H. Felis or H. mustelis may produce gastric diseases in animal models.

Cross reactivity between proteins from Helicobacter species may mean that antigens from an individual bacterial species could provide protection in an animal which is not its normal host.

The dominant antigens to which antibody is detected in Helicobacter pylori-negative individuals are the 18-19 and 24-25 kDa antigens. Hence, use of an antigenic preparation containing all antigens less than 30 kDa, preferably less than 29, ideally less than 28 and preferably less than 27 kDa and would be enriched in the immunodominant antigens to be used in putative vaccine.

It will be apparent that cytokine interleukin 12 acts as an adjuvant to potentiate the immunogenicity of H. pylori. In particular, it potentiates the immunogenicity of protein fractions of less than 30 kDa, especially the 18 kDa and 25 kDa protein fractions of H. pylori.

It will be appreciated that interleukin 12 may also be used as an adjuvant with any H. pylori protein or derivative or fragment thereof. Its application is not limited to the specific 25 kDa or 18 kDa proteins referred to above. The interleukin 12 may be conjugated with the H. pylori unit in such a way as to allow the interleukin to be released in vivo, for example by peptic acid and gastric enzymes/or urease.

It will be appreciated by those skilled in the art that while we have referred to a molecular mass of 24 to 25 kDa and 18 to 19 kDa the molecular mass may lie in the 24-26 kDa and 17-19 kDa range. Other related organisms such as H. Felis or H. mustelis may produce gastric diseases in animal models.

Cross reactivity between proteins from Helicobacter species may mean that antigens from an individual bacterial species could provide protection in an animal which is not its normal host.

The dominant antigens to which antibody is detected in Helicobacter pylori-negative individuals are the 18-19 and 24-25 kDa antigens. Hence, use of an antigenic preparation containing all antigens less than 30 kDa, preferably less than 29, ideally less than 28 and preferably less than 27 kDa and would be enriched in the immunodominant antigens to be used in putative vaccine.

Partial sequencing of the two antigens from Helicobacter pylori

N-terminal Sequence Analysis

Purified 18 and 24 kDa proteins were electroblotted to PVDF and ProBlott, respectively, from 12.5% polyacrylamide gels. The proteins were located on the membranes by staining with 0.1% amido black (in 1% acetic acid, 40% method) for 15s followed by destaining in several changes of distilled deionized water. The membranes were air-dried thoroughly and submitted for sequence analysis using the Edman degradation procedure as described by Matsudaira (198921).

The N-terminal amino acid sequence of the 25 and 18 kDa protein are given in Sequence Id No's 1 and 2 respectively.

Peptide Mapping

The N-chlorosuccinimide peptide mapping method of Lischwe and Ochs (1982)²² was used with minor modifications. Bands of interest were located on SDS-PAGE gels (12.5% T) by briefly staining the gel with 0.1% Coomassie Blue R250 (in 50% methanol, 10% acetic acid) and then excised with a scalpel blade. The protein present in the gel slices was digested with N-chlorosuccinimide (15 mM) in acetic acid/urea/water (1:1:1, v/w/v) for 30 min at 20° C. The treated gel slices were then washed with several changes of water and equilibrated with SDS-PAGE sample buffer exactly as described by Lischwe and Ochs. Finally, the gel slices were placed in the sample wells of a 15% polyacrylamide SDS-PAGE gel and electrophoresed. Following electrophoresis, the separated peptides were transferred to either PVDF or ProBlott by Western blotting. Peptides were visualized by staining the membrane with 0.1% amido black in acetic acid (1%) and methanol (40%). After extensive washing with water, the peptides were submitted for sequencing without any further modifications.

Mercaptoacetic acid (2 mM) was included in the upper electrode buffer during all SDS-PAGE electrophoretic procedures. This mobile thiol behaves as a free radical scavenger and thus prevents N-blocking.

Amino acid sequences for internal peptides from the 18 and 25 kDa protein are given in Sequence Id. No.'s 3 and 4 respectively.

Extraction of Helicobacter pylori Chromosomal DNA

Chromosomal DNA was extracted as described (Silhavy et al., 1984. Experiments with gene fusions. C.S.H. publications).

Amplifying the sequence of the 18-19 protein kDa gene of using degenerate primers.

Degenerate DNA sequence was deduced from the amino acid sequences listed in Sequence Id. No.'s 2 and 3. Four degenerate primers were designed from these sequences, to allow for a two stage, nested, PCR reaction. Eag1 restriction enzyme sites were built into each primer, to allow for subsequent cloning of the fragment. Where three or more bases were possible at any site, inosine was incorporated instead of all possible bases, except, where such sites were four bases or less from the primers 3′ (3 prime) terminal, in which case all possible bases were included. Inosine was also avoided at positions immediately adjacent to the Eag1 sites.

Degenerate primers for gene p18:

1. GAARA CGGCC GARAT IYTIA ARCAY YTICA RGC

2. TCYTC GGCCG TYTCY TCIGT NGCY

3. RATIY TCGGC CGYYI CARGC IGAYG C

4. ATYTC GGCCG TIGCY TTRTG NAC

Genomic DNA for the 18-19 kDa protein gene p18 was amplified as follows using the outer set of primers (primers 1 & 2): the samples were heated to 94 degrees C for 3 minutes to denature the DNA, followed by 35 cycles of 94 degrees C for 30 seconds, 56 degrees C for 40 seconds and 72 degrees C for 30 seconds. 100 pmol of each primer was used, in the presence of 2.5 mM MgCl₂ and 0.2 mM dNTPs, in a reaction volume of 50 ul. 1 ul of this reaction was used as the substrated for the ‘nested’ reaction. This reaction was the same as outlined for the above reaction, except that the inner primers (primers 3 & 4) were substituted for the external primers, and a concentration of 2.0 mM MgCl₂ was used. Electrophoresis of the products of the reaction resulted in a clearly visible band on a 2% agarose gel, estimated at approximately 120 bp in size (as judged by a molecular size ladder).

Sequencing the Amplified DNA Sequence.

The nested PCR fragment corresponding to the 18-19 kDa protein gene was cloned by digesting the fragment with Eag1 and ligating this into the unique Eag1 site in the Bluescript vector (Stratagene). E. coli cells were transformed (according to standard procedures) and plasmid DNA was harvested using the alkaline lysis method (Sambrook et al., 1989. Molecular cloning: A laboratory manual 2nd. Ed., CSH publications) followed by an RNAase digestion step, phenol/chloroform extraction and precipitation using 2.5M ammonium acetate and 2 volumes of ethanol. Two independent isolates of plasmid DNA were sequenced using forward and reverse universal sequencing primers. The inserted DNA derived from the pl8 gene was sequenced in the forward and reverse orientations. Sequencing was performed using an ABI automated sequencer and a Genpak PCR based fluorescent dideoxy chain terminator termini sequencing kit.

The sequence of bases between the terminal of the internal PCR primers is:

GATCGTGTTATTTATGAAAGTGCATAACTTCCATTGGAATGTGAAAGGCAC CGATTTTTTCAAT

This sequence of bases translates into the amino acid sequence listed in Sequence Id. No. 5.

This sequence (Sequence Id. No. 5) overlaps with both the 18 kDa protein N-terminal amino acid sequence listed in Sequence Id. No. 2 and the 18 kDa protein internal amino acid sequence listed in Sequence No. 3, to give the enlarged N-terminal amino acid sequence listed in Sequence Id. No. 6.

Many variations on the specific embodiments described will be readily apparent and accordingly the invention is not limited to the embodiments hereinbefore described which may be varied in detail.

### LIST OF REFERENCES

1. Marshall, B. J. and Warren, J. R. (1984). Unidentified curved bacilli in the stomach of patients with gastritis and peptic ulceration. Lancet 1, 1311-1314.

2. Blaser M. J. (1990). Helicobacter pylori and the pathogenesis of gastrodudodenal inflammation. J. Infect. Dis. 161, 626-633.

3. Rauws, E. A. J. and Tytgat, G. N. J. (1990). Eradication of Helicobacter pylori cures duodenal ulcer: Lancet 1, 1233-1235.

4. Lambert, J. R., Borromeo, M., Pinkard, K. J., Turner, H., Chapman, C. B., and Smith, M. L. (1987). Colonisation of gnotobiotic pigs with Campylobacter pylori—an animal model ? J. Infect. Dis. 155, 1344.

5. Marshall, B. J., Armstrong, J. A., McGechie, D. B., and Glancy, R. J. (1985). Attempt to fulfil Koch's postulates for pyloric Campylobacter. Med. J. Aust. 142, 436-439.

6. Morris, A. and Nicholson, G. (1987). Ingestion of Campylobacter pylori causes gastritis and raises fasting gastric pH. Am. J. Gastroenterol. 82, 192-199.

7. Jiang, S. J., Liu, W. Z., Zhang, D. Z., Shi, Y., Xiao, S. D., Zhang, Z. N., and Liu, D. Y. (1987). Campylobacter-like organisms in chronic gastritis, peptic ulcer and gastric carcinoma. Scand. J. Gastroenterol. 22, 553-558.

8. Lambert, J. R., Dunn, K. A., Eaves, E. R., Korman, M. G., and Hansky, J. (1986). Campylobacter pyloridis in diseases of the human upper gastrointestinal tract. Gastroenterology 90, 1509.

9. Crabtree, J. E., Figura, N., Taylor, J. D., Bugnoli, M., Armellini, D., and Tompkins, D. S. (1992). Expression of 120 kDa protein and cytotoxicity in Helicobacter pylori. J. Clin. Pathol. 45, 733-734.

10. Crabtree, J. E., Wyatt, J. I., Sobala, G. M., Miller, G., Tompkins, D. S., Primrose, J. N., and Morgan, A. G. (1993). Systemic and mucosal humoral responses to Helicobacter pylori in gastric cancer. Gut 34, 1339-1343.

11. Forman, D., Sitas, F.,. and Newell, D. G. (1990). Geographic association of Helicobacter pylori antibody prevalence and gastric cancer mortality in rural China. Int. J. Cancer 46, 608-611.

12. Forman, D., Newell, D. G., Fullerton, F., Yarnell, J. W. G., Stacey, A. R., Wald, N., and Sitas, F. (1991). Association between infection with Helicobacter pylori and risk of gastric cancer: evidence from a prospective investigation. BMJ 302, 1302-1305.

13. Nomura, A., Stemmermann, G. N., Chyou, P-H., Kato, I., Perez-Perez, G. I., and Blaser, M. J. (1991). Helicobacter pylori infection and gastric carcinoma amongst Japanese Americans in Hawaii, N. Engl. J. Med. 325, 1132-1136.

14. Parsonnet, J., Friedman, G. D, Vandersteen, D. P., Chang, Y., Vogelman, J. H., Orentreich, N., and Sibley, R. K. (1991). Helicobacter pylori infection and the risk of gastric carcinoma. N. Engl. J. Med. 325, 1127-1131.

15. Forman, D. (1993). An international association between Helicobacter pylori infection and gastric cancer. The EUROGAST Study Group. Lancet 341, 1359-1362.

16. Blaser, M. J. (1992). Hypothesis on the pathogenesis and natural history of Helicobacter pylori-induced inflammation. Gastroenterology 102, 720-727.

17. Taylor, D. N. and Blaser, M. J. (1991). Epidemiology of Helicobacter pylori infection. Epidemiol. Rev. 13, 42-59.

18. Fan, X. J., Chua, A. Shahi, C. N., McDevitt, J., Keeling, P. W. N. and Kelleher, D. (1994) Gastric T lymphocyte responses to Helicobacter pylori colonisation. Gut 35, 1379-1384.

19. Laemmli, U. K. (1970). Nature 227, 680-685.

20. Markwell, M. A. K., Haas, S. M., Bieber, L. L. and Tolbert, N. E. (1978) Analytical Biochemistry, 87, 206-210.

21. Matsudaira, P. T. (1989). A practical guide to protein and peptide purification for microsequencing. Academic Press, San Diego.

22. Lischwe, M. A. and Ochs, D. (1982). A new method for partial peptide mapping using N-chlorosuccinimide/urea and peptide silver staining in sodium dodecyl sulfatepolyacrylamide gels. Analytical Biochemistry 127, 453-457.

1. A vaccine including at least one Helicobacter protein or derivative or fragment or precursor or mutant thereof to which immunoreactivity is detected in H. pylori negative individuals.

2. A vaccine as claimed in claim 1 wherein the immunoreactivity is antibody based.

3. A vaccine as claimed in claim 1 wherein the protein is a Helicobacter pylori protein.

4. A vaccine as claimed in claim 1 wherein the protein is a single protein or a mixture of proteins having a molecular weight of less than 30 kDa, preferably less than 29 kDa, most preferably less than 28 kDa, particularly less than 27 kDa, and especially a 24 to 25 kDa protein or a derivative or fragment or precursor or mutant thereof.

5. A vaccine as claimed in claim 4 wherein the 24 to 25 kDa protein, has an N-terminal amino acid sequence listed in Sequence Id. No. 1, or a portion thereof, preferably the 24 to 25 kDa protein has an internal amino acid sequence listed in Sequence Id. No. 4, or a portion thereof.

6. A vaccine as claimed in claim 1 wherein the protein includes an 18 to 19 kDa protein, or a derivative, fragment or precursor or mutant thereof.

7. A vaccine as claimed in claim 6, wherein the 18 to 19 kDa protein, has an N-terminal amino acid sequence listed in Sequence Id. No. 2, or a portion thereof, preferably wherein the 18 to 19 kDa protein, has an internal amino acid sequence listed in Sequence Id. No. 3, or a portion thereof, preferably wherein the 18 to 19 kDa protein has an N-terminal amino acid sequence listed in Sequence Id. No. 6, or a portion thereof.

8. A vaccine as claimed in claim 1 in combination with a pharmacologically suitable adjuvant, especially interleukin 12 and/or the adjuvant is a heat shock protein.

9. A vaccine as claimed in claim 1 wherein the vaccine includes at least one other pharmaceutical product such as an antibiotic which may be selected from one or more of metronidazole, amoxycillin, tetracycline or erythromycin, clanithromycin or tinidazole, alternatively or additionally the pharmaceutical product includes an antibacterial agent such as bismuth salts.

10. A vaccine as claimed in claim 1 in a form for oral administration, intranasal administration, intravenous administration, intramuscular administration or including a peptide delivery system.

11. A vaccine as claimed in claim 1 for the treatment or prophylaxis of Helicobacter pylori infection or Helicobacter pylori associated disease.

12. A Helicobacter protein or derivative or fragment or precursor or mutant thereof to which immunoreactivity is detected in H. pylori negative individuals.

13. A Helicobacter protein as claimed in claim 12 wherein the immunoreactivity is antibody based.

14. A Helicobacter protein as claimed in claim 12 which is a Helicobacter pylori protein.

15. A Helicobacter pylori protein as claimed in claim 14, wherein the protein has a molecular weight of less than 30 kDa, preferably less than 29 kDa, most preferably less than 28 kDa, particularly less than 27 kDa, and especially a 24 to 25 kDa protein or a derivative or fragment or precursor or mutant thereof.

16. A Helicobacter pylori protein as claimed in claim 15 wherein the 24 to 25 kDa protein, has an N-terminal amino acid sequence listed in Sequence Id. No. 1, or a portion thereof, preferably the 24 to 25 kDa protein, has an internal amino acid sequence listed in Sequence Id. No. 4, or a portion thereof.

17. A Helicobacter pylori protein as claimed in claim 14 wherein the protein is an 18 to 19 kDa protein, or a derivative, fragment or precursor or mutant thereof.

18. A Helicobacter pylori protein as claimed in claim 17, wherein the 18 to 19 kDa protein, has an N-terminal amino acid sequence listed in Sequence Id. No. 2, or a portion thereof, preferably wherein the 18 to 19 kDa protein, has an internal amino acid sequence listed in Sequence Id. No. 3, or a portion thereof, preferably wherein the 18 to 19 kDa protein has an N-terminal amino acid sequence listed in Sequence Id. No. 6, or a portion thereof.

19. A method for the treatment or prophylaxis of Helicobacter pylori associated disease in a host comprising administering a Helicobacter pylori protein as claimed in claim 14.

20. A method as claimed in claim 19 wherein the Helicobacter pylori protein is administered in combination with at least one other pharmaceutical agent such as an antibiotic which may be selected from one or more of metronidazole, amoxycillin, tetracycline erythromycin, clarithromycin or tinidazole, and/or a pharmaceutical agent including an antibacterial agent such as bismuth salts.

21. A method as claimed in claim 19 wherein the Helicobacter pylori protein is administered in combination with an adjuvant, the adjuvant preferably being interleukin 12 and/or a heat shock protein.

22. Monoclonal or polyclonal antibodies or fragments thereof, to the proteinaceous material of claim 14.

23. Purified antibodies or serum obtained by immunisation of an animal with the vaccine according to claim 14.

24. A vaccine for the treatment or prophylaxis of Helicobacter pylori associated disease comprising an immunogenically effective amount of a Helicobacter pylori protein of claim 14 an adjuvant such as Interleukin 12, and an antibiotic.

25. A vaccine against H. pylori comprising an immunogenically effective amount of a Helicobacter or a subunit, fragment, derivative, precursor or mutant thereof in combination with interleukin 12 as an adjuvant.

26. A vaccine as claimed in claim 25 wherein the Helicobacter is Helicobacter pylori, preferably the vaccine includes an antibiotic and/or an antibacterial agent.
